(19) United States
(12) Patent Application Publication
(10) Pub. No.: US 2014/0186970 A1
(43) Pub. Date: Jul. 3, 2014

(54) IMPRINTED PHOTONIC POLYMERS AND METHODS FOR THEIR PREPARATION AND USE

(71) Applicant: Xiaobin Hu, Shanghai (CN)

(72) Inventor: Xiaobin Hu, Shanghai (CN)

(73) Assignee: EMPIRE TECHNOLOGY DEVELOPMENT LLC, WILMINGTON, DE (US)

(21) Appl. No.: 13/817,105

(22) PCT Filed: Nov. 2, 2011
(86) PCT No.: PCT/CN11/81678

§ 371 Date: Feb. 14, 2013

(51) Int. Cl. G01N 33/20

(52) U.S. Cl. CPC G01N 33/20
USPC 436/501; 521/189; 422/69

(57) ABSTRACT

Macroporous matrices containing molecularly imprinted photonic polymers (MIPPs) and methods of making these macroporous matrices are disclosed herein. The macroporous matrices can, for example, be used for detection of small molecules, such as metal ions, in a sample.

### BACKGROUND

1. Field

The present application relates to compositions and methods for detecting small molecules, such as metal ions, in a sample.

2. Description of the Related Art

The presence of metal ions, for example lead ions, in various water bodies, soils, crops and foods, has become a global environmental problem. Some commonly used methods and instruments allow sensitive and specific detections of metal ions; however, their use is limited by disadvantages such as complicated sample pretreatments, time-consuming operations, and high instrumental and operational costs. There is a need for fast and low-cost methods for detecting metal ions in a sample with high selectivity and sensitivity.

### SUMMARY

Some embodiments enclosed herein include a macroporous matrix for detecting a metal ion in a sample, wherein the matrix comprises molecularly imprinted photonic polymers (MIPPs), wherein the MIPPs comprise at least one binding cavity specific for the metal ion. In some embodiments, the binding cavity comprises one or more binding sites for the metal ion.

In some embodiments, the macroporous matrix has an average pore size of about 150 nm to about 400 nm. In some embodiments, the macroporous matrix is interconnected. In some embodiments, the macroporous matrix has the form of a bead, gel, membrane, particle, film, or combinations thereof. In some embodiments, the film has a thickness of about 2 μm to about 100 μm. In some embodiments, the macroporous matrix is attached to a solid support. In some embodiments, the solid support is glass, nylon, paper, nitrocellulose, plastic, or combinations thereof. In some embodiments, the MIPPs comprise chitosan polymers, polyethylene glycol polymers, copolymers of chitosan and polyethylene glycol, vinyl polymers, or combinations thereof. In some embodiments, the vinyl polymers are poly(4-vinylbenzo-18-crown-6), poly(N-methacryloyl-cysteine), poly(vinyl benzoate), or combinations thereof.

In some embodiments, the metal ion is a heavy metal ion. In some embodiments, the metal ion is Pb²⁺, Cu²⁺, Hg²⁺, Cd²⁺, Cr³⁺, Cr⁶⁺ or combinations thereof. In some embodiments, the metal ion is Pb²⁺.

Some embodiments enclosed herein include a method of preparing a macroporous matrix for detecting a metal ion in a sample, the method includes: (a) providing a colloid crystal template, wherein the colloid crystal template comprises an array of colloidal crystals on a solid support; (b) contacting the metal ion with at least one monomer under conditions to allow the metal ion to bind the monomer; (c) forming a first composition comprising the colloidal crystal template and the monomer that bound with the metal ion; (d) maintaining the first composition under conditions to allow polymerization of the monomers and imprinting of the metal ion to form a second composition; and (e) removing the colloid crystal template and the metal ion from the second composition to prepare the macroporous matrix.

In some embodiments, the colloidal crystals are polymeric colloids, inorganic colloids, metal colloids, ceramic colloids, coated colloids, semiconductor colloids, or combinations thereof. In some embodiments, the colloidal crystals are silica colloidal crystals, polystyrene (PS) colloidal crystals, methyl methacrylate (PMMA) colloidal crystals, or combinations thereof. In some embodiments, the colloidal particles are silica colloidal crystals. In some embodiments, the colloidal crystals comprise colloid particles having an average diameter of about 150 nm to about 400 nm. In some embodiments, the silica colloidal crystals comprise colloid particles having an average diameter of about 200 nm.

In some embodiments, the monomer comprises at least one amino group, at least one hydroxyl group, at least one carboxyl group, or combinations thereof. In some embodiments, the solid support is glass, nylon, paper, nitrocellulose, plastic or combinations thereof. In some embodiments, the metal ion binds to the monomer by chelation. In some embodiments, the monomer is chitosan, polyethylene glycol, or a vinyl monomer. In some embodiments, the vinyl monomer is 4-vinylbenzo-18-crown-6,N-methacryloyl-cysteine, or vinyl benzoate.

In some embodiments, the maintaining step is performed in the presence of a polymerization initiator. In some embodiments, the polymerization initiator is 2,2-azobis isobutyronitrile (AIBN), azoimide, or benzoyl peroxide. In some embodiments, the maintaining step is performed in the presence of a crosslinking agent. In some embodiments, the crosslinking agent is glutaraldehyde. In some embodiments, the maintaining step is performed with ultraviolet light irradiation.

In some embodiments, the removing step comprises contacting the second composition with an eluent. In some embodiments, the eluent is hydrofluoric acid or toluene.

Some embodiments disclosed herein include a method for detecting a metal ion from a sample, the method include: providing a sample suspected of containing the metal ion; contacting the sample with a macroporous matrix, wherein the matrix comprises molecularly imprinted photonic polymers (MIPPs), wherein the MIPPs comprise at least one binding cavity specific for the metal ion; and detecting a change of the macroporous matrix. In some embodiments, the change is a colorimetric change.

In some embodiments, the detecting step is carried out by an optical sensor.

In some embodiments, the detecting step is carried out by naked eye observation of a user. In some embodiments, the colorimetric change of the macroporous matrix is correlated with the concentration of the metal ion in the sample. In some embodiments, the concentration of the metal ion in the sample is about 0.1 nM to about 10 mM.

In some embodiments, the metal ion is a heavy metal ion. In some embodiments, the metal ion is Pb²⁺, Cu²⁺, Hg²⁺, Cd²⁺, Cr³⁺, Cr⁶⁺ or combinations thereof. In some embodiments, the metal ion is Pb²⁺.

Some embodiments disclosed herein include an apparatus for detecting a metal ion in a sample, the apparatus includes: at least one light source; and a receiver configured to receive at least a portion of the radiation emitted from the light source, wherein the receiver comprises a macroporous matrix, wherein the matrix comprises molecularly imprinted photonic polymers (MIPPs), wherein the MIPPs comprise at least a binding cavity specific for the metal ion. In some embodiments, the apparatus further comprises at least one light detector configured to measure light emitted from or absorbed by the receiver. In some embodiments, the light source is configured to emit an ultraviolet or violet radiation.

### BRIEF DESCRIPTION OF THE DRAWINGS

FIG. 1 is a schematic diagram illustrating an embodiment of a method for detecting metal ions using a macroporous matrix containing MIPPs through a colorimetric change that is within the scope of the present application. FIG. 1A shows a macroporous matrix and its color and band stop prior to binding with the metal ions. FIG. 1B shows the macroporous matrix and its color and band stop after binding with the metal ions.

FIG. 2 depicts an illustrative embodiment of an apparatus for detecting metal ions that is within the scope of the present application (not to scale).

FIG. 3A-F is a schematic diagram illustrating an embodiment of a preparation process of a macroporous matrix containing Pb²⁺-imprinted MIPPs that is within the scope of the present application.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Disclosed in the present application are macroporous matrices containing molecularly imprinted photonic polymers (MIPPs) for detecting small molecules, such as metal ions, in a sample. As described in the present application, the MIPPs are polymers prepared by the photonic crystal technique in combination with the molecular imprinting technique, which include at least one binding cavity specific for the target small molecules. The macroporous matrices containing MIPPs can provide, in some embodiments, highly sensitive, selective and rapid detection of small molecules, including metal ions (e.g., lead ions Pb²⁺). The present application also relates to methods of making these macroporous matrices, method of using these macroporous matrices, and apparatuses for detecting small molecules that include these macroporous matrices.

### Macroporous Matrix Containing Molecularly Imprinted Photonic Polymer (MIPP)

### Molecularly Imprinted Polymer

Molecularly imprinted polymers (MIPs) are polymers with selective adsorption capability for target molecules which are prepared by molecular imprinting technique. Molecular imprinting creates specific recognition sites for target molecules in substrate materials, such as polymeric organic materials. Preparation of molecularly imprinted polymers typically involves mixing target molecules (that is, the molecule to be imprinted) with a functional monomer or a mixture of functional monomers to form imprint/monomer complexes, where the target molecules interact or bond with a complementary portion of a functional monomer through covalent, ionic, hydrophobic, hydrogen-bonding or other interactions. The imprint/monomer complexes are then polymerized and/or crosslinked into a polymeric matrix. The target molecules are subsequently dissociated (e.g., cleaved) from the functional monomers and thereby removed from the polymer matrix to leave “cavities” in the polymer matrix, where the cavities have morphologies and sizes substantially similar to those of the target molecules and/or specific recognition sites for the target molecule. In general, molecularly imprinted polymers are in a gel or polymeric mold-like structure having multiple molecular-scale cavities that are complementary to the target molecules, which gives the ability to specifically bind the target molecules.

The methods of polymerization of MIPs around a template entity have been described in various references such as Peter A. G. Cormack et al., Journal of Chromatography B, 804 (2004) 173-182 (describing various techniques available around aspects of MIP polymerization), U.S. Pat. No. 4,127,730 (describing a covalent approach for molecular imprinting), and U.S. Pat. No. 5,110,833 (describing a noncovalent approach for molecular imprinting). The covalent and noncovalent approaches can be combined for synthesizing MIPs. For example, as disclosed in Whitcombe et al. “A new method for the introduction of recognition site functionality into polymers prepared by molecular imprinting: synthesis and characterization of polymeric receptors for cholesterol,” J. Am. Chem. Soc., 117:7105-7111 (1995), it is possible to use the covalent-type approach for the preparation of the MIP and the noncovalent-type approach for obtaining recognition of the target molecule by means of noncovalent interactions. As disclosed in Wulff et al. Macromol. Chem. Phys. 190:1717, 1727 (1989), it is also possible to combine the covalent-type and noncovalent-type approaches for the preparation of the MIP and for obtaining the recognition by means of covalent and noncovalent interactions simultaneously for the same target molecule. As a result, the interaction occurs at least at two distinct sites of the recognition site. In addition, a “semi-covalent” approach for the synthesis of the MIPs is described in U.S. Patent Publication No. 20100234565.

Various target molecules can be used as imprinting targets to generate molecularly imprinted polymers. For example, small molecules such as drugs; stimulants; organic chemicals; and metal ions, such as Cu^{2+, Ni}²⁺, Cd²⁺, Co²⁺, Hg²⁺, Pb²⁺ and ions of noble metal and lanthanide series metals, can be used as imprinting targets to prepare corresponding MIP.

### Molecularly Imprinted Photonic Polymer (MIPP)

Photonic crystal polymers, such as photonic hydrogels and photonic ionic liquid polymers, are capable of responding to various stimuli such as pH, metal ions, glucose, creatinine and anions with high sensitivity. Upon response to various chemical stimuli, the photon band gap offset of the photonic crystal polymers is induced, which can result in the color change of the photonic crystal polymers change, and subsequently allows for detecting various chemical stimuli by using colorimetric methods. However, photonic polymers are usually universal rather than specific responsive, especially to those chemical stimuli coming from molecular or ionic analogs, and thus they are generally incompetent as highly specific chemosensors for analyte detections.

As disclosed in the present application, photonic crystal polymers can be used in conjunction with the molecular imprinting technique to prepare molecularly imprinted photonic polymers (MIPPs). MIPPs are MIPs fabricated on a template prepared from colloidal crystals, such as silica colloidal crystals. The porous nature of the colloidal crystal template allows infiltration of the polymerizable monomers, target molecules and the bound monomer-target molecules into the void spaces of the colloidal crystal template and in situ polymerization of the monomers within the void spaces. After etching the colloidal crystals and eluting imprinted target molecules, the MIPPs form a macroporous polymeric matrix. In some embodiments, the polymeric matrix containing MIPPs comprises an ordered three-dimensional macroporous structure. In some embodiments, the macroporous matrix containing MIPPs has an inversed opal structure. For example, the macroporous matrix has at least one macropore resulted from the removal of the colloidal crystals and at least one molecular-scaled cavity (i.e., nanocavity) that has morphological appearance and size substantially similar to those of the target molecule. In some embodiments, at least two of the macropores in the macroporous matrix are connected. In some embodiments, the macroporous matrix is interconnected. The target molecule-shaped nanocavities are capable of selectively receiving target molecules, and thus can direct the target molecule in a sample to the selective binding site therein. In some embodiments, the macroporous matrix containing MIPPs has at least one residual nanocavity specifically accessible for the target molecule. In some embodiments, the macroporous matrix responds to a chemical stimulus by a readable optical signal.

As used herein, the term “binding cavity” refers to a molecular-scaled cavity in the macroporous matrix containing MIPPs which has a morphological appearance and size substantially similar to those of the target molecule. In some embodiments, the binding cavity is specific to the target molecule. As used herein, the term “binding site” refers to a site that exists in the binding cavities of the macroporous matrix containing MIPPs which can specifically bind to a target molecule, such as a metal ion. In some embodiments, the binding cavity comprises one binding site for the target molecule. In other embodiments, the binding cavity comprises two or more binding sites for the target molecule. The binding interaction between the target molecule and the binding site is not limited in any way. Non-limiting examples of the binding interaction include the formation of weak bonds, for example, the van der Waals bonds, hydrogen bonds, pidonor-pi acceptor bonds, and hydrophobic interactions; and the formation of strong bonds, for example, the ionic bonds, covalent bonds, and iono-covalent bonds.

The macroporous matrices disclosed in the present application can include various polymers, such as chitosan polymers, polyethylene glycol polymers, copolymers of chitosan and polyethylene glycol, vinyl polymers, acrylic polymers, and acrylamide polymers or combinations thereof. In some embodiments, the vinyl polymers are poly(4-vinylbenzo-18-crown-6), poly(N-methacryloyl-cysteine), poly(vinyl benzoate), poly(vinylpyridine), poly(vinylimidazole), or combinations thereof. In some embodiments, the macroporous matrix comprises chitosan polymers, polyethylene glycol polymers, and copolymers of chitosan and polyethylene glycol. In some embodiments, the macroporous matrix comprises poly(vinylpyridine). Without being bound to any particular theory, it is believed that the polymers (such as chitosan polymers) contain a large quantity of functional groups, for example, amino groups, hydroxyl groups, and carboxyl groups; and therefore chelation, for example strong chelation, can occur between the functional groups in the polymer (such as chitosan) and the metal ion (such as lead ion). And without being bound to any particular theory, it is believed that polyethylene glycol can form a crown ether-like structure matching with the metal ion through change of molecular conformation, and these interactions can improve the sensitivity of the macroporous matrix responding to the target molecule such as a metal ion, including a lead ion, as well as improve the selectivity of the response.

In some embodiments, the macroporous matrix containing MIPPs has an average pore size of about 50 nm to 1000 nm, about 100 nm to about 800 nm, about 120 nm to about 600 nm, about 140 nm to about 500 nm, about 150 nm to about 400 nm, about 170 nm to about 350 nm, about 190 nm to about 300 nm, or about 180 nm to about 250 nm. In some embodiments, the macroporous matrix has an average pore size of about 50 nm, about 100 nm, about 150 nm, about 200 nm, about 250 nm, about 300 nm, about 350 nm, about 400 nm, about 450 nm, about 500 nm, about 600 nm, about 700 nm, about 800 nm, and ranges between any two of these values. In some embodiments, the macroporous matrix has an average pore size of an average diameter of about 150 nm to about 400 nm. In some embodiments, the macroporous matrix has an average pore size of about 200 nm.

The macroporous matrix containing MIPPs can be in various forms. For example, the macroporous matrix can be in the form of a bead, gel, membrane, particle, fiber, foil, film, or combinations thereof. In some embodiments, macroporous matrix is in the form of a bead, gel, membrane, particle, film, or combinations thereof. In some embodiments, the macroporous matrix is in the form of a film, for example, a porous polymer film. In some embodiments, the macroporous matrix is in the form of a hydrogel. In some embodiments, macroporous matrix is in the form of a film. The thickness of the film is not limited in anyway. For example, the thicknesses of the film can be about 0.1 μm to about 1000 μm, about 0.5 μm to about 500 μm, about 1 μm to about 300 μm, about 1.5 μm to about 200 μm, about 2 μm to about 100 μm, about 5 μm to about 80 μm, about 10 μm to about 50 μm, or about 20 μm to about 40 μm. In some embodiments, the thicknesses of the film can be about 0.1 μm, about 0.5 μm, about 1 μm, about 1.5 μm, about 2 μm, about 5 μm, about 10 μm, about 20 μm, about 50 μm, about 100 μm, about 150 μm, about 200 μm, and ranges between any two of these values.

In some embodiments, the macroporous matrix containing MIPPs is attached to a solid support. Examples of solid support include, but are not limited to glass, nylon, paper, nitrocellulose, plastic, or combinations thereof.

Some embodiments disclosed in the present application include a macroporous matrix for detecting metal ions, wherein the macroporous matrix comprises molecularly imprinted photonic polymers (MIPPs), wherein the MIPPs comprise at least one binding cavity specific for the metal ion. In some embodiments, the metal ion is a heavy metal ion. In some embodiments, the metal ion is Pb²⁺, Cu²⁺, Hg²⁺, Cd²⁺, Cr³⁺, Cr⁶⁺, or combinations thereof. In some embodiments, the metal ion is Pb²⁺.

### Method of Making a Macroporous Matrix Containing MIPPs for Detecting Metal Ions

Some embodiments disclosed herein include a method of making a macroporous matrix for detecting metal ions, wherein the matrix comprises molecularly imprinted photonic polymers (MIPPs), wherein the MIPPs comprise at least one binding cavity specific for the metal ion.

As disclosed above, MIPPs are synthesized using photonic crystal polymers in conjunction with the molecular imprinting technique. MIPPs are molecularly imprinted polymers fabricated on a template prepared from colloidal crystals, such as silica colloidal crystals. For example, colloids can be used to prepare a colloidal crystal template which allows infiltration of the polymerizable monomers, target molecules and the bound monomer-target molecules into its void spaces and allows in situ polymerization of the monomers in the void spaces. After etching the colloidal crystals and eluting imprinted target small molecules, in some embodiments, the MIPPs form a macroporous polymeric matrix in an inversed opal structure. In some embodiments, the macroporous matrix has at least one macropore resulted from the removal of the colloidal crystals and at least one binding cavity that has a morphological appearance and size substantially similar to those of the target molecule.

In some embodiments, the method for making a macroporous matrix containing MIPPs includes: (a) providing a colloid crystal template, wherein the colloid crystal template comprises an array of colloidal crystals on a solid support; (b) contacting the metal ion with at least one monomer under conditions to allow the metal ion to bind the monomer; (c) forming a first composition comprising the colloidal crystal template and the monomer that bound with the metal ion; (d) maintaining the first composition under conditions to allow polymerization of the monomers and imprinting of the metal ion to form a second composition; and (e) removing the colloid crystal template and the metal ion from the second composition to prepare the macroporous matrix. In some embodiments, colloids are deposited onto a solid support to prepare the colloid crystal template. Various support substrates can be used as the solid support, for example, glass, metallic surface, nylon, paper, nitrocellulose, plastic, PTFE, methyl methacrylate (PMMA), mixed cellulose esters, polycarbonate, polypropylene, and combinations thereof. In some embodiments, the solid support is glass, nylon, paper, nitrocellulose, plastic or combinations thereof. In some embodiments, the solid support is glass. In some embodiments, the solid support is PMMA.

Any suitable colloidal particles of any shape can be used in the methods and compositions disclosed in the present application. The colloidal particles can be chosen depending upon the optimum degree of ordering and the resulting lattice spacing desired for the particular application. Colloids (i.e., colloidal particles) can be made from materials, including, but are not limited to, inorganic substrate such as silica and alumina, polymeric materials such as polystyrene (PS) and poly(methyl methacrylate) (PMMA), and metals such as transition metals, post-transition metals and semiconductors. Colloids can comprise a single material such as silica or alumina, or a combination of materials including, but not limited to, a combination of metals, inorganic substances or polymeric materials. Colloids can be prepared using techniques known in the art. In some embodiments, colloids are polymeric colloids, inorganic colloids, metal colloids, ceramic colloids, coated colloids, semiconductor colloids, or combinations thereof. In some embodiments, colloids are silica colloids, polystyrene colloids, poly(methyl methacrylate) (PMMA) colloids, or combinations thereof.

The colloids can be a homogenous or heterogeneous mixture. When comprises of a single material, the colloids are homogenous. When comprises of a combination of materials, the colloids can be a homogenous mixture of the combination of materials, or the different materials can be separated into different regions of the colloids. For example, a colloid comprising a polymer and an inorganic material can have the inorganic material at the core and the polymeric material on the exterior of the colloid. One of skill in the art will appreciate the colloids having at least two layers of materials are useful in the compositions and methods disclosed herein, and that the composition and thickness of each layer can be adjusted to meet the need of the desired application.

100361 Various sizes of colloidal particles can be used. For example, the average diameter of the colloidal particles can be about 50 nm to 1000 nm, about 100 nm to about 800 nm, about 120 nm to about 600 nm, about 140 nm to about 500 nm, about 150 nm to about 400 nm, about 170 nm to about 350 nm, about 190 nm to about 300 nm, or about 180 rim to about 250 nm. The colloidal particles can have an average diameter of about 50 nm, about 100 nm, about 150 nm, about 200 nm, about 250 nm, about 300 nm, about 350 nm, about 400 nm, about 450 nm, about 500 nm, about 600 nm, and ranges between any two of these values. In some embodiments, the colloid particles have an average diameter of about 150 nm to about 400 nm. In some embodiments, the colloidal particles are silica colloidal particles. In some embodiments, the silica colloidal particles have an average diameter of about 200 nm.

Examples of solvents for preparing mixtures of colloids include, but are not limited to, water, alcohols (such as ethanol and propanol) and any polar, protic solvent. The solution of colloids can have concentration from about 0.1% to about 99%, from about 0.5% to about 50%, from about 0.8% to about 40%, from about 1% to about 30%, from about 2% to about 20%, from about 3% to about 10%, or from about 5% to about 8% by mass percentage. The solution of colloids can have concentration of about 0.1%, about 0.5%, about 1%, about 1.5%, about 2%, about 5%, about 10%, about 20%, about 30%, and ranges between any two of these values, by mass percentage. In some embodiments, the solution of colloids has a concentration of about 1% by mass percentage.

In some embodiments, crystallization of colloids into colloidal crystals is accomplished by promoting the evaporation of the solvent used to deposit the colloids onto the solid support. The conditions used for the crystallization step can depend on the solvent used, the type of the colloids, the size of the colloids, the concentration of the colloid solution, the temperature during crystallization, as well as other factors apparent to one of skill in the art. Examples of solvents for the crystallization of the colloidal crystals include, but are not limited to, water, alcohols (such as ethanol and propanol), and any polar, protic solvent. One of skill in the art will appreciate that other variables including pH, salt concentration of the solvent, pressure can be varied to generate arrays of colloidal crystals having desired characteristics.

Examples of colloid crystals include, but are not limited to, silica colloid crystals, polystyrene (PS) colloidal crystals, poly(methyl methacrylate) (PMMA) crystals, and any combinations thereof. The shape of the colloidal crystals is not limited in anyway. For example, a colloidal crystal can be in the shape of square, round, elliptical, triangular, rectangular, polygonal and toroidal. In some embodiments, the colloid crystals are silica colloid crystals.

Various polymerizable monomers can be used to bind the target molecule, for example the metal ion, in the compositions and methods described in the present application. In some embodiments, the monomer has at least one binding site for the metal ion. In some embodiments, the monomer has at least two binding sits for the metal ion. In some embodiments, the monomer has at least three binding sits for the metal ion. The type of binding between the metal ion and the monomer is not limited in any way, for example, the binding can be covalent or noncovalent bonding. In some embodiments, the metal ion binds to the monomer by chelation. Examples of monomers that can be used to bind the metal ion include, but are not limited to, chitosan; polyethylene glycol; acrylics; acrylamides; vinyl monomers with chelating groups (for example, crown group, sulfhydryl group, carboxyl group, and amido group) in side chain, such as 4-vinylbenzo-18-crown-6,N-methacryloyl-cysteine, vinyl imidazole, vinyl pyridine, and vinyl benzoate. In some embodiments, the monomer comprises at least one functional group capable of chelating with the metal ion. In some embodiments, the monomer comprises at least two functional groups capable of chelating with the metal ion. In some embodiments, the monomer comprises at least one amino group, at least one hydroxyl group, at least one carboxyl group, at least one crown group, or combinations thereof. In some embodiments, the functional group(s) in the monomer serve as chelating-ligand to bind the metal ion, allowing formation of a stable chelate compound of the monomer with the metal ion.

In some embodiments, the colloidal crystal template and the monomer that bound with the metal ion are maintained in conditions suitable for polymerization of the monomers. For example, the monomers can be polymerized in the presence of a polymerization initiator. Non-limiting examples of the polymerization initiator include 2,2-azobis isobutyronitrile (AIBN), azoimide, peroxide (such as benzoyl peroxide), and combinations thereof. The monomers can be polymerized in the presence of a crosslinking agent. Non-limiting examples of crosslinking agent include glutaraldehyde, oxalaldehyde, ethyleneglycol dimethacrylate, and combinations thereof. In some embodiments, the monomers are polymerized with ultraviolet light irradiation.

Variant eluents can be used to remove the colloid crystal template and the metal ion(s) to prepare the macroporous matrix containing MIPPs as described in the present application. In some embodiments, at least one eluent is used. In some embodiments, at least two eluents are used. In some embodiments, the eluent for removing colloid crystal template is the same as the eluent for removing the metal ions(s). In some embodiment, the eluent for removing colloid crystal template is different as the eluent for removing the metal ions(s). Non-limiting examples of eluent include hydrofluoric acid, toluene, and chloroform. In some embodiments, the colloidal crystal template comprises silica colloidal crystals and the eluent is hydrofluoric acid. In other embodiments, the colloidal crystal template comprises PS and/or PMMA and the eluent is toluene. In some other embodiments, the colloidal crystal template comprises PS and the eluent is chloroform.

The macroporous matrix containing MIPPs can be used on any appropriate support. The support can be any flexible or rigid solid substrate on or in which MIPPs are capable of being bound, adhesively bounded, deposited, synthesized in-situ, filled and/or packaged. The support can be of any nature, for instance of biological, nonbiological, organic or inorganic nature, or a combination thereof. The support can be in any form, for example, the forms of particles, gels, sheets, tubes, spheres, capillaries, tips, films or wells, of any size or any shape. For example, the macroporous matrix containing MIPPs can be deposited and/or used on or in a support chosen from a multi-well plate, a strip, a paper, a chip, a glass, a silica plate, a thin layer, a porous surface, a nonporous surface, a microfluidic system. In some embodiments, the macroporous matrix containing MIPPs is deposited and/or used on a glass. In some embodiments, the macroporous matrix containing MIPPs is deposited and/or used on a cellulose substrate.

### Methods and Apparatuses for Detecting Metal Ions

Some embodiments of the present application include methods and apparatuses for detecting small molecules, such as metal ions from a sample.

A method for detecting small molecules, such as metal ions, can include providing a sample suspected of containing the metal ion and contacting the sample with a macroporous matrix described in this application. In some embodiments, the binding of the metal ion to the macroporous matrix induces a change in the photonic and/or structural property of the macroporous matrix. The change can be detected using any means known in the art. The photonic property of the macroporous matrix which that can be used to detect the presence of the metal ion in a sample includes, but is not limited to, the stop band property, the gap band property or the dispersion property. In some embodiments, the change in the macroporous matrix is a change in the volume. In some embodiments, the change in the macroporous matrix is a change in the shape. In some embodiments, the change is a colorimetric change. In some embodiments, the change is a structural change.

In some embodiments, the stop band of the macroporous matrix containing MIPPs is used to detect the metal ion in the sample. In some embodiments, the band offset of the macroporous matrix containing MIPPs is used to detect the metal ion in the sample. The stop band and the change in the stop band following binding of the metal ion to the macroporous matrix containing MIPPs can be detected by, e.g., measuring reflected light or transmitted light by the macroporous matrix. One of skill in the art will appreciate that the macroporous matrix comprising different types of materials will have different stop bands. In some embodiments, binding of the metal ion to the macroporous matrix containing MIPPs induces a shift in the stop band or stop band peak of at least about 1, about 5, about 10, about 15, about 20, about 30, about 40, about 50, about 60, about 70, about 80, about 90, about 100, about 150, about 200, about 250, about 300 nm as compared to the stop band or stop band peak in the macroporous matrix without binding of the metal ion.

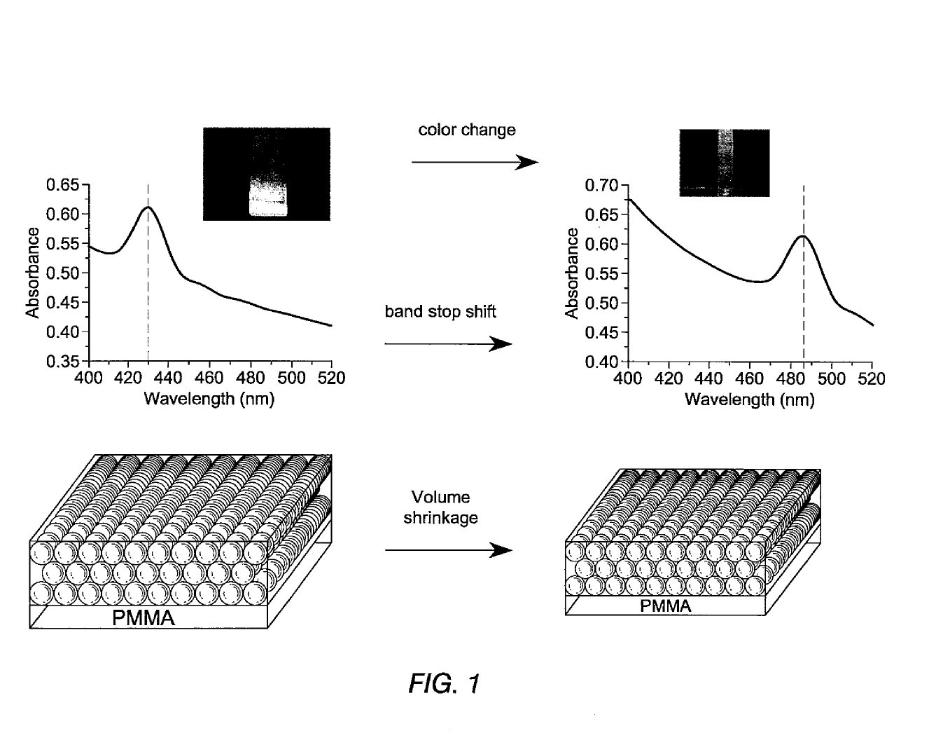

The macroporous matrices disclosed in the present application can selectively bind metal ions, such as Pb²⁺. As schematically illustrated in FIG. 1, in some embodiments, the binding of metal ions, such as Pb²⁺, leads to expansion or contraction in volume of the macroporous matrix. In some embodiments, the volume change in the macroporous matrix causes the offset of the band gap of the photonic crystal structure to produce color change, which allows colorimetric detection of the metal ions. The macroporous matrices disclosed in the present application can be used to detect the presence of metal ions, such as Pb²⁺, as well as to measure the concentration of the metal ions. In some embodiments, the response of the macroporous matrix containing MIPPs to the presence of a metal ion, such as Pb²⁺, and/or the concentration (or the change in concentration) of the metal ion, is converted into a detectable signal. In some embodiments, the detectable signal is an optical signal, such as a color change of the macroporous matrix. In some embodiments, the color change is detectable by naked eye observation of a user or an optical sensor. In some embodiments, the optical signal is detected by an ultraviolet-visible spectrophotometer.

In some embodiments, the structural property of the macroporous matrix containing MIPPs is used to detect the presence of the metal ion in the sample. In some embodiments, the macroporous matrix changes its shape or volume. In some embodiments, the macroporous matrix swells or deflates. The expansion or shrinkage in the entire or a partial portion of the macroporous matrix can be measured using interferometetric method. In some embodiments, binding of the metal ion to the macroporous matrix induces swelling or deflation of the macroporous matrix by at least about 1%, about 5%, about 10%, about 15%, about 20%, about 30%, about 40%, about 50% or more in volume as compared to the macroporous matrix in the absence of the metal ion.

Non-limiting examples of metal ions that can be detected using the compositions and methods disclosed in the present application include heavy metal ions, noble metal ions, nutritious metal ions, and ions of rare earth metal. Examples of heavy metal ion include As³⁺, As⁵⁺, Cd²⁺, Cr⁶⁺, Pb²⁺, Sb³⁺, Sb⁵⁺, Ni²⁺, Ag⁺ and Tl³⁺. In some embodiments, the metal ion is Cu²⁺, Ni²⁺, Cd²+, Co²⁺, Hg²⁺, Ca²⁺, or Pb²⁺. In some embodiments, the metal ion is Cu²⁺, Cd²⁺, Cr³⁺, Cr⁶⁺, Hg²⁺, or Pb²⁺. In some embodiments, the metal ion is Pb²⁺.

The macroporous matrix described in the present application can be used for detecting metal ions in various types of samples. In some embodiments, the sample is an environmental sample, a food sample, a biological sample. In some embodiments, the sample is an aqueous sample. Examples of aqueous sample include, but are not limited to ocean water, wastewater, blood, urine, sewage, plant discharge, groundwater, polluted river water, industrial waste, battery waste, electroplating wastewater, liquid waste in chemical analysis, and laboratory waste. In some embodiments, the wastewater is generated from industrial factories such as printery, non-ferrous metal manufacturing, mining, smelting, electrolysis, electroplating, chemicals, medicine, paint and pigment. In some embodiments, the untreated sample is automotive exhaust.

The compositions and methods described herein allow detection of metal ions in a wide range of concentrations, including very low concentrations. For example, the concentration of the metal ion in the sample can be about 10⁻¹² mol/L (10⁻¹²M) to about 10 mM (10⁻³ M), about 10⁻¹¹ M to about 10⁻⁴ M, about 10⁻¹⁰ M to about 10⁻⁵ M, and about 10⁻⁹ M to about 10⁻⁶ M. The concentration of the metal ion in the sample can be about 10⁻¹³ M, 10⁻¹² M, about 10⁻¹¹ M, about 10⁻¹⁰ M, about 10⁻⁹ M, about 10⁻⁸ M, about 10⁻⁷ M, about 10⁻⁶ M, about 10⁻⁵ M, about 10⁻⁴ M, about 10⁻³ M, about 10⁻² M, and ranges between any two of these values. In some embodiments, the concentration of the metal ion in the sample is less than about 10⁻⁸ M. In some embodiments, the concentration of the metal ion in the sample is about 10⁻⁹ M. In some embodiments, the concentration of the metal ion in the sample is about 10⁻¹⁰ M. In some embodiments, the concentration of the metal ion in the sample is about 10⁻¹¹ M. In some embodiments, the concentration of the metal ion in the sample is about 10⁻¹² M. In some embodiments, the concentration of the metal ion in the sample is about 10⁻¹³ M.

The compositions and methods described herein can also allow rapid detection of metal ions. For example, the minimal time needed for the sample to contact with the macroporous matrix containing MIPPs to allow detection of the metal ion and/or measuring of the metal ion concentration can be about 60 minutes, about 50 minutes, about 40 minutes, about 30 minutes, about 20 minutes, about 10 minutes, about 5 minutes, about 4 minute, about 3 minutes, about 2 minutes, about 1 minutes, about 0.5 minute, about 0.2 minute, about 0.1 minute, or shorter, or ranges between any two of these values. In some embodiments, the minimal time needed for the sample to contact with the macroporous matrix containing MIPPs to allow detection of the metal ions and/or measuring of metal ion concentration is at most about 1 second, at most about 3 seconds, at most about 6 seconds, at most about 9 seconds, at most about 12 seconds, at most about 18 seconds, at most about 24 seconds, at most about 30 seconds, at most about 1 minute, at most about 5 minutes, or at most 10 minutes, or ranges between any two of these values.

Some embodiments of the present application include apparatuses for detecting small molecules, such as metal ions from a sample. In some embodiments, the apparatus includes: at least one light source; and a receiver configured to receive at least a portion of the radiation emitted from the light source, wherein the receiver comprises a macroporous matrix described in the present application. In some embodiments, the light source provides an intensity and wavelength sufficient to excite the macroporous matrix. Suitable light sources are known to those of skill in the art and are commercially available.

In some embodiments, the apparatus further comprises at least one light detector configured to measure light emitted from or absorbed by the receiver. In some embodiments, the light source is configured to emit an ultraviolet or violet radiation. In some embodiments, the apparatus further comprises a housing, wherein the housing contains the macroporous matrix containing MIPPs and is configured to receive a sample adjacent to the macroporous matrix containing MIPPs. For example, the macroporous matrix containing MIPPs can be exposed to the light source such as a laser at a preset angle of incidence before, during, and/or after contacting the macroporous matrix with a sample suspected of containing the metal ion. A change in the stop band, stop band peak, or index of refraction indicates binding of the metal ion to the macroporous matrix. The light detector can be an optical sensor adapted to detected light emitted from the macroporous matrix.

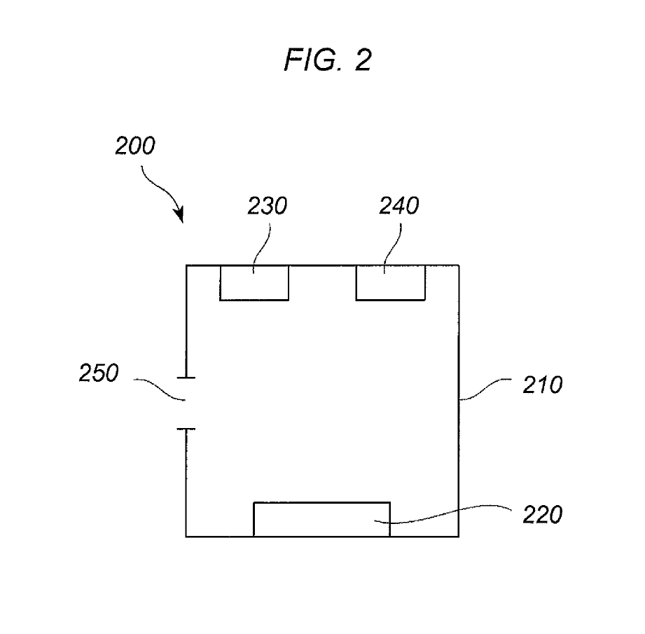

FIG. 2 depicts an illustrative embodiment of an apparatus for detecting a target molecule that is within the scope of the present application. Apparatus 200 can include housing 210 that contains a macroporous matrix containing MIPPs 220, light source 230, light detector 240, and port 250. Light source 230 is configured to emit radiation effective to produce fluorescence from macroporous matrix 220. For example, light source 230 can be an InGaN semiconductor that emits blue or ultraviolet radiation. Light detector 240 can be configured to measure light emission from or light adsorption by macroporous matrix 220. Port 250 can be configured to receive a sample into the housing. Thus, for example, a sample suspected of containing one or more target molecules, such as lead ions, can be placed into housing 210 via port 250, so that the sample contacts macroporous matrix 220. Light source 230 can then emit light and the absorption by or reflectance from macroporous matrix 220 is detected by light detector 240. The amount of the absorption or reflectance can then be correlated with the presence of the target molecule, such as a lead ion, in the sample.

### EXAMPLES

Additional embodiments are disclosed in further detail in the following examples, which are not in any way intended to limit the scope of the claims.

### Example 1

### Preparation of Pb²⁺-Imprinted Photonic Polymer

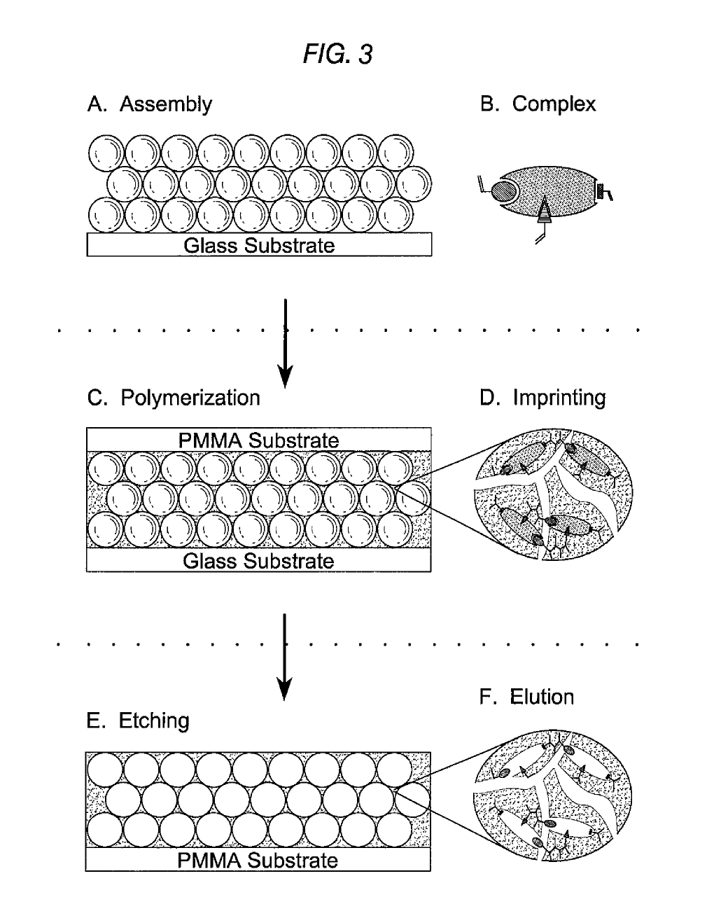

The preparation process for Pb²⁺-imprinted photonic polymer film is illustrated in the flow chart shown in FIG. 3.

### a) Preparation of Silica Colloidal Crystal

Silica colloidal particles with average particle diameter of about 200 nm are dispersed in absolute ethanol solution. Silica colloidal crystals are generated on a clean glass substrate via colloid particle self-assembly under temperature-constant humidity-constant conditions (FIG. 3-a).

### b) Chelation of Lead Ions and Monomers

Chitosan and polyethylene glycol are used as polymer functional monomer for forming a hydrogel. Pb(NO₃)₂ is used as the Pb²⁺ source of imprinting. The chitosan and polyethylene glycol monomers are dispersed and mixed with Pb(NO₃)₂ under ultrasonic wave in an acidic solution of pH=4-6 for 4 hours to allow adequate chelation of Pb²⁺ and the monomers in an aqueous solution. A schematic illustration of the resulting complex in which lead ions are chelated with chitosan and polyethylene glycol monomer is shown in FIG. 3-b.

### c) Monomer Polymerization and Lead Ion Imprinting

Polymerization initiator 2,2-azobis isobutyronitrile (AIBN) and crosslinking agent glutaraldehyde are mixed into the acidic aqueous solutions containing Pb²⁺, chitosan and polyethylene glycol monomers under ultrasonic wave to initiate polymerization and crosslink of chitosan and polyethylene glycol. The mixture is then added dropwise into the silica colloid crystal template prepared in step a) until the template becomes transparent, and the colloid crystal template is covered with a clean organic glass plate, such as a PMMA substrate. The colloid crystal plate having adsorbed the aqueous solution of Pb²⁺-chelated monomers is polymerized under an ultraviolet lamp light for a polymerization time period of 1-3 hours. Chitosan and polyethylene glycol are crosslinked in the presence of glutaraldehyde to form a solid polymer film, in which the silica colloid crystals are embedded. The schematic illustrations of the polymerization and imprinting processes are shown in FIG. 3-c, d.

### d) Removal of Colloid Crystal Template and Elution of Lead Ions

The solid polymer film obtained in step c) is soaked in a 4% hydrofluoric acid solution (by mass percentage) for about 1 hour to remove the embedded silica colloid crystals. The resulting porous polymer film is rinsed with 1 M hydrochloric acid until Pb²⁺ is undetectable in the rinsed liquid, which indicates that Pb²⁺ has been completely eluted from the polymer film. The polymer film is then rinsed with ultrapure water and 0.1 M phosphate buffer solution of pH=7.4 for several times until the film is neutral.

As illustrated in FIG. 3-e, f, the porous polymer contains many macropores created by the removal of the silica colloid crystals, as well as a large number of cavities with morphological appearances and sizes substantially matching with those of Pb²⁺ (that is, Pb²⁺-imprinted nano-cavities). The interconnective macroporous structure of the porous polymer is favorable for ion diffusion, which allows for quick and sensitive response to the target metal ion in a sample. These properties endow the polymer film with high affinity and selectivity to Pb²⁺.

### Example 2

### Establish Standard for Measuring Lead Ion Concentration by Pb²⁺-Imprinted Photonic Polymer

The lead ion-imprinted three-dimensional photonic polymer is prepared according to the procedure described in Example 1. The porous polymer is spread on colorless transparent organic glass plates to prepare test papers. A set of aqueous solutions with known and different concentrations of lead ions is provided. Each piece of test paper is inserted into a lead ion solution in the solution set. The colorimetric response of each test paper is measured using an ultraviolet-visible spectrophotometer. The position of band gap of each of the test paper is recorded. It is expected that the band gap position of the test paper and the lead ion concentration are correlated, and thus the band gap position is indicative of the concentration of the lead ion in the sample. Accordingly, the positions of band gap recorded for the sample set with known lead ion concentrations can be used as a standard for measuring lead ions concentration.

### Example 3

### Detection of Lead Ions Using Pb²⁺-Imprinted Photonic Polymer

The lead ion-imprinted three-dimensional photonic polymer is prepared according to the procedure described in Example 1. The porous polymer is spread on a colorless transparent organic glass plate to prepare a test paper. The test paper is inserted into an aqueous sample suspected of containing Pb²⁺. The positions of band gap of the test paper prior to and after being inserted into the sample are measured using an ultraviolet-visible spectrophotometer. A shift in the position of band gap indicates the presence of Pb²⁺ in the sample.

### Example 4

### Measurement of Pb²⁺ Concentration Using Pb²⁺-Imprinted Photonic Polymer

The lead ion-imprinted three-dimensional photonic polymer is prepared according to the procedure described in Example 1. The porous polymer is spread on a colorless transparent organic glass plate to prepare a test paper. The test paper is inserted into an aqueous sample with unknown Pb²⁺ concentration. The position of band gap of the test paper is measured using an ultraviolet-visible spectrophotometer. The Pb²⁺ concentration in the sample is determined by comparing the position of the band gap measured and the standard for measuring lead ion concentration that is established according to the procedure described in Example 2.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A macroporous matrix for detecting a metal ion in a sample, wherein the matrix comprises molecularly imprinted photonic polymers (MIPPs), wherein the MIPPs comprise at least one binding cavity specific for the metal ion.

2. The macroporous matrix of claim 1, wherein the binding cavity comprises one or more binding sites for the metal ion.

3. (canceled)

4. The macroporous matrix of claim 1, wherein the macroporous matrix is interconnected.

5. The macroporous matrix of claim 1, wherein the macroporous matrix has the form of a bead, gel, membrane, particle, film, or combinations thereof.

6. (canceled)

7. (canceled)

8. The macroporous matrix of claim 1, wherein the macroporous matrix is attached to a solid support.

9. The macroporous matrix of claim 8, wherein the solid support is glass, nylon, paper, nitrocellulose, plastic, or combinations thereof.

10. The macroporous matrix of claim 1, wherein the MIPPs comprise chitosan polymers, polyethylene glycol polymers, copolymers of chitosan and polyethylene glycol, vinyl polymers, or combinations thereof.

11. The macroporous matrix of claim 10, wherein the vinyl polymers are poly(-vinylbenzo-18-crown-6), poly(N-methacryloyl-cysteine), poly(vinyl benzoate), or combinations thereof.

12. The macroporous matrix of claim 1, wherein the metal ion is a heavy metal ion.

13. The macroporous matrix of claim 1, wherein the metal ion is Pb²⁺, Cu²⁺, Hg²⁺, Cd²⁺, Cr³⁺, Cr⁶⁺ or combinations thereof.

14. (canceled)

15. A method of preparing a macroporous matrix for detecting a metal ion in a sample, the method comprising:
providing a colloid crystal template, wherein the colloid crystal template comprises an array of colloidal crystals on a solid support;
contacting the metal ion with at least one monomer under conditions to allow the metal ion to bind the monomer;
forming a first composition comprising the colloidal crystal template and the monomer that bound with the metal ion;
maintaining the first composition under conditions to allow polymerization of the monomers and imprinting of the metal ion to form a second composition; and
removing the colloid crystal template and the metal ion from the second composition to prepare the macroporous matrix.

16. The method of claim 15, wherein the colloidal crystals are polymeric colloids, inorganic colloids, metal colloids, ceramic colloids, coated colloids, semiconductor colloids, or combinations thereof.

17. The method of claim 15, wherein the colloidal crystals are silica colloidal crystals, polystyrene (PS) colloidal crystals, methyl methacrylate (PMMA) colloidal crystals, or combinations thereof.

18. (canceled)

19. The method of claim 15, wherein the colloidal crystals comprise colloid particles having an average diameter of about 150 nm to about 400 nm.

20. (canceled)

21. The method of claim 15, wherein the monomer comprises at least one amino group, at least one hydroxyl group, at least one carboxyl group, or combinations thereof.

22. The method of claim 15, wherein the solid support is glass, nylon, paper, nitrocellulose, plastic or combinations thereof.

23. The method of claim 15, wherein the metal ion binds to the monomer by chelation.

24. The method of claim 15, wherein the monomer is chitosan, polyethylene glycol, or a vinyl monomer.

25. The method of claim 24, wherein the vinyl monomer is 4-vinylbenzo-18-crown-6,N-methacryloyl-cysteine, or vinyl benzoate.

26. The method of claim 15, wherein the maintaining step is performed in the presence of a polymerization initiator.

27. (canceled)

28. The method of claim 15, wherein the maintaining step is performed in the presence of a crosslinking agent.

29. (canceled)

30. The method of claim 15, wherein the maintaining step is performed with ultraviolet light irradiation.

31. The method of claim 15, wherein the removing step comprises contacting the second composition with an eluent.

32. (canceled)

33. A method for detecting a metal ion from a sample, the method comprising:
providing a sample suspected of containing the metal ion;
contacting the sample with a macroporous matrix, wherein the matrix comprises molecularly imprinted photonic polymers (MIPPs), wherein the MIPPs comprise at least one binding cavity specific for the metal ion; and
detecting a change of the macroporous matrix.

34. The method of claim 33, wherein the change is a colorimetric change.

35. The method of claim 33, wherein the detecting step is carried out by an optical sensor.

36. (canceled)

37. The method of claim 33, wherein the colorimetric change of the macroporous matrix is correlated with the concentration of the metal ion in the sample.

38. The method of claim 33, wherein the concentration of the metal ion in the sample is about 0.1 nM to about 10 mM.

39. The method of claim 33, wherein the metal ion is a heavy metal ion.

40. The method of claim 33, wherein the metal ion is Pb²⁺, Cu²⁺, Hg²⁺, Cd²⁺, Cr³⁺, Cr⁶⁺ or combinations thereof.

41. (canceled)

42. An apparatus for detecting a metal ion in a sample, the apparatus comprising:
at least one light source; and
a receiver configured to receive at least a portion of the radiation emitted from the light source, wherein the receiver comprises a macroporous matrix, wherein the matrix comprises molecularly imprinted photonic polymers (MIPPs), wherein the MIPPs comprise at least a binding cavity specific for the metal ion.

43. The apparatus of claim 42, further comprising at least one light detector configured to measure light emitted from or absorbed by the receiver.

44. The apparatus of claim 41, wherein the light source is configured to emit an ultraviolet or violet radiation.
